(19) United States
(12) Patent Application Publication
(10) Pub. No.: US 2015/0366462 A1
(43) Pub. Date: Dec. 24, 2015

(54) Temperature Sensing Catheter

(71) Applicant: C.R. Bard, Inc., Murray Hill, NJ (US)

(72) Inventors: Ruben Ramos, Loganville, GA (US); David Icenogle, Atlanta, GA (US)

(21) Appl. No.: 14/764,969

(22) PCT Filed: Mar. 12, 2014
(86) PCT No.: PCT/US14/24886

§ 371 Date: Jul. 30, 2015

Related U.S. Application Data
(60) Provisional application No. 61/794,849, filed on Mar. 15, 2013.

(51) Int. Cl. A61B 5/01; B05D 1/18; A61B 5/00

(52) U.S. Cl. CPC A61B 5/01; A61B 5/6853; A61B 5/0008; A61B 5/742; B05D 1/18; A61B 2562/12

(57) ABSTRACT

An improved catheter is described. The catheter may have an inflation lumen reinforced with a metal support, such as a coil, to prevent collapse and deflation of the inflation lumen, while leaving a minimal impact on the size of the catheter. The catheter may be manufactured with a temperature sensing strip permanently integrated into the catheter during the manufacturing process. The temperature sensing strip is able to wirelessly send information regarding a patient's temperature to an external display, where it may be available for viewing by a care provider. Additionally, the drainage lumen of the catheter is preferably coated with a hydrophobic coating or treatment, and/or formed to include a patterned microstructure surface design, such as superhydrophobic patterned surface.

### PRIORITY

This application claims the benefit of priority to U.S. Provisional Application No. 61/794,849, filed Mar. 15, 2013, which is incorporated by reference in its entirety into this application.

### BACKGROUND

The present invention relates generally to medical catheters, and particularly to catheters and methods for reinforcing an inflation lumen, and also measuring a patient's core body temperature and wirelessly transmitting the measurements to an external display.

Foley catheters are generally tubes having a rounded tip at a distal end that is inserted into the bladder of a patient, and a proximal end that remains outside the body of the patient. Foley catheters are typically utilized to remove urine from the bladder of a patient. A Foley catheter generally includes a balloon disposed at a distal end of the catheter to anchor the catheter in the bladder, the catheter also including at least one drainage lumen to drain urine from the bladder and at least one inflation lumen to inflate the balloon (e.g., with sterile water). The proximal end of the Foley catheter can include two ports in communication with the two lumens (i.e., the drainage lumen and the inflation lumen). A first port connected to the drainage lumen can have an interface with fittings for drainage and sampling, and a second port connected to the inflation lumen can have a valve to ensure the inflation fluid remains within the lumen and balloon once filled. The tip of a Foley catheter extends beyond the sides of the balloon into the bladder and includes one or more apertures or “eyes” to drain fluids and debris from the bladder once the tip is positioned inside the bladder.

Foley catheters can have issues with deflation once they are inside a patient. This can be due to a variety of factors that cause the balloon's inflation lumen to collapse. An inappropriate insertion of inflation fluid may result in an improperly inflated inflation lumen due to under-inflation (e.g., adding an insufficient amount of inflation fluid to a larger inflation balloon) and non-aspiration of the syringe (e.g., not properly loosening or preparing the syringe for insertion of a fluid). Also, a balloon is under abnormally high radially inward pressure. This radially inward pressure can result from any number of causes, including but not limited to, under-inflation of the balloon, anatomical abnormality, and excessive traction resulting from physician placement or patient movement The radially inward pressure exerted on the balloon results in a radially inward pressure exerted on the catheter shaft, which causes the outer surface of the catheter to push into the inflation lumen, closing or very nearly closing off the inflation lumen.

In addition, when a negative pressure is exerted by a syringe trying to aspirate fluid from the balloon, the effect can be to completely collapse the walls of the inflation lumen, making it difficult or impossible to deflate the balloon. Thus, even if the inflation lumen is properly inflated, collapse of the inflation lumen during removal and consequent balloon deflation results in ridges or cuff formation which can result in urethral trauma and make atraumatic removal of the catheter difficult or impossible. On occasion, it proves difficult or impossible to deflate the balloon in the normal manner. When this happens, it becomes necessary to take extraordinary means such as inserting an instrument up the catheter through the inflation lumen or through the bladder to pierce the balloon to allow the inflation medium to escape. These procedures may cause the patient additional discomfort and may lead to adverse clinical consequences.

Some Foley catheters include a temperature sensor included on the end of the catheter. A wire connects the sensor, via the catheter, to externally located monitoring devices. Use of a temperature-sensing catheter allows for convenient and continuous temperature monitoring, helping to maintain a normal body temperature. It also maintains a closed system and eliminates invasive probes to maximize patient safety. This type of Foley catheter typically has a thermistor or thermocouple located on or near the tip of the device and a wire that runs the length of the catheter to a connector that plugs into a temperature monitor. In some instances an additional external cable is also used, which may or may not be removable. However, current methods of manufacturing a temperature-sensing catheter can be costly and tedious, and patients in hospitals are usually inundated with an inordinate amount of tubing. Further, a Foley catheter with a temperature sensor cannot be connected to an external cable and/or the temperature monitor if the temperature sensor has not been shown to be safe for patients undergoing MRI examinations.

### SUMMARY

Accordingly, described herein are urinary catheters including features believed to provide advantages over existing Foley catheters. In one embodiment, a urinary catheter includes a temperature sensor, wirelessly sending core body temperature data to an external display. In one embodiment, a method of manufacturing a catheter includes integrating a wireless temperature sensor during the manufacturing process. In one embodiment, a method of manufacturing a catheter includes integrating a reinforced metal support in the inflation lumen. In one embodiment, a urinary catheter includes an inflation lumen reinforced with a metal support, such as a metal braid or coil, along a portion or all of its length.

In one embodiment, a catheter includes a proximal end and a distal end, a balloon disposed near the distal end proximal of a tip formed at the distal end, a drainage lumen extending from a drainage eye in the side wall of the tip to the proximal end, the drainage lumen including a superhydrophobic microstructure patterned surface, an inflation lumen extending from an inflation eye near the distal end in fluid communication with the balloon to the proximal end of the catheter, the inflation lumen including a reinforcement member, and a temperature sensor disposed at the distal end of the catheter proximal the drainage eye.

In one embodiment, a catheter includes a proximal end and a distal end, a balloon disposed near the distal end proximal a tip formed at the distal end, a drainage lumen extending from a drainage eye in the side wall of the tip to the proximal end, an inflation lumen extending from an inflation eye near the distal end in fluid communication with the balloon to the proximal end of the catheter, and a temperature sensor disposed at the distal end of the catheter proximal the drainage eye.

In one embodiment, a catheter includes a catheter including a proximal end and a distal end, a balloon disposed near the distal end proximal a tip formed at the distal end, a drainage lumen extending from a drainage eye in the side wall of the tip to the proximal end, and an inflation lumen extending from an inflation eye near the distal end in fluid communication with the balloon to the proximal end of the catheter, the inflation lumen including a reinforcement member.

In one embodiment, a method of forming a catheter includes dipping an inflation wire, drainage form, and temperature sensor individually in a first coating material, and dipping an inflation wire, drainage form, and temperature sensor longitudinally aligned together in a second coating material.

In one embodiment, a method of forming a catheter includes dipping a reinforced inflation wire and drainage form individually in a first coating material, and dipping an inflation wire and drainage lumen longitudinally aligned together in a second coating material.

These and other embodiments, methods, features and advantages will become more apparent to those skilled in the art when taken with reference to the following more detailed description of the invention in conjunction with the accompanying drawings that are first briefly described.

### BRIEF DESCRIPTION OF THE DRAWINGS

The disclosed systems and methods can be better understood with reference to the following drawings. The components in the drawings are not necessarily to scale.

FIG. 1 shows a cross-section of a distal end of a catheter in accordance with the present disclosure.

FIG. 2 shows an aspect of a method of manufacturing a catheter in accordance with the present disclosure.

FIG. 3 shows a side view of a catheter in accordance with the present disclosure.

FIG. 4 shows an aspect of a method of manufacturing a catheter in accordance with the present disclosure.

FIG. 5 shows an exemplary superhydrophobic microstructure patterned surface formed in a drainage lumen in accordance with the present disclosure.

### DESCRIPTION

The following description and accompanying figures, which describe and show certain embodiments, are made to demonstrate, in a non-limiting manner, several possible configurations of a catheter according to various aspects and features of the present disclosure.

For clarity it is to be understood that the word “proximal” as used herein refers to a direction relatively closer to a clinician, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

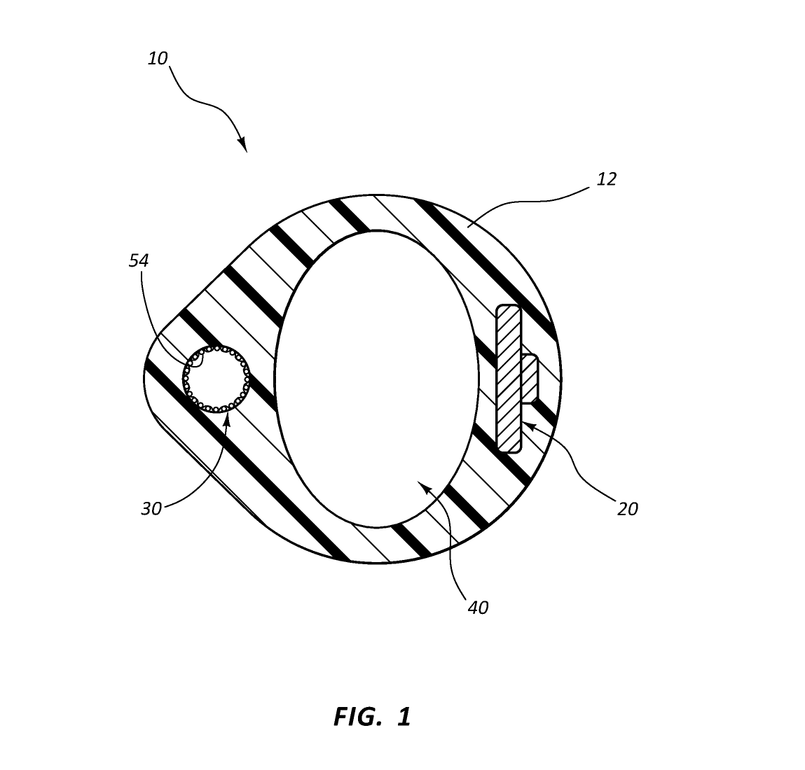

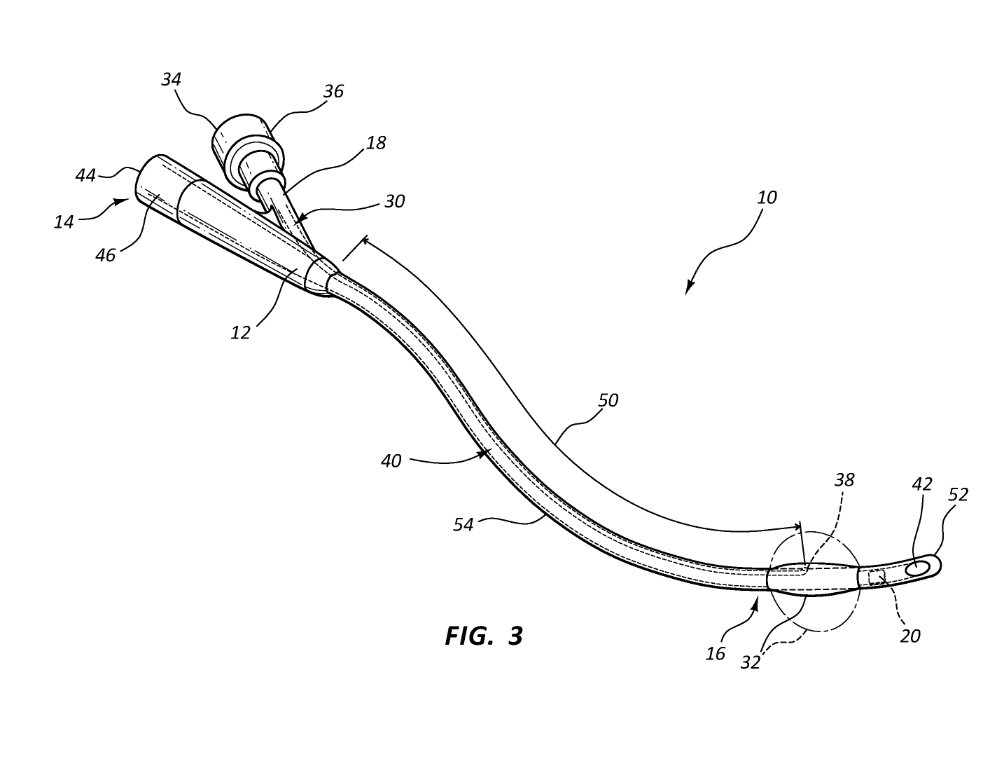

Referring to FIG. 1, a distal end 16 of catheter 10 is illustrated in cross-section with an inflation lumen 30, drainage lumen 40, and temperature sensor 20. The catheter 10 comprises an elongated catheter body 12. As shown in FIG. 1, the inflation lumen 30 may include a reinforcement 54 as described in more detail below (e.g., with a metal braided material). As shown in FIG. 3, catheter 10 has a proximal end 14 and a distal end 16. A balloon 32 is located near the distal end 16 of the catheter adjacent the tip 52 of the catheter 10. The catheter tip 52 may have a rounded, atraumatic end. A drainage lumen 40 extends longitudinally within the catheter body 12 from proximal end 14 to drainage eye(s) 42 in the side wall(s) of tip 52, and is in fluid communication with drainage eye(s) 42. Although a single drainage eye 42 is illustrated, it is contemplated that the tip 52 may include multiple drainage eyes 42. Drainage eye(s) 42 permit fluid to enter the drainage lumen 40. Drainage eye(s) 42 may be burnished and polished for added smoothness to maximize patient comfort. Drainage eye(s) 42 may be relatively large holes to reduce clotting and maximize urine flow.

The drainage lumen 40 comprises a major portion of the cross-section of the central region of catheter body 12. The proximal end 14 of the drainage lumen 40 is placed in fluid communication with fluid collection or disposal equipment, such as a urinary drainage bag. The proximal end 14 of catheter 10 may include a drainage port 44 in fluid communication with the drainage lumen 40. Optionally, the proximal end 14 of catheter 10 may include a one-way drainage valve 46 that only allows fluid to drain proximally from the catheter 10, and prevents reflux of drained urine back into the catheter 10. Also, proximal end 14 of catheter 10 may include or be attached to other communication valves, chambers, funnels, or other devices through which the drainage lumen 40 communicates and/or attaches to the fluid collection or disposal equipment.

The inflation lumen 30 is formed within the wall of the catheter body 12 and extends from an inflation eye 38 inside of the balloon 32 to the proximal end 14 of catheter body 12. Catheter body 12 may include a branching arm 18 in a proximal region of the catheter body 12 through which the inflation lumen 30 passes. In use, balloon 32 is inflated once the distal end 16 of catheter 10 is positioned within a bladder of the body of the patient, which serves to anchor the distal end 16 in the bladder. The proximal end 14 of catheter 10 may include an inflation port 34 in fluid communication with the inflation lumen 30 of the catheter 10. Optionally, the proximal end 14 of catheter 10 may also include an inflation valve 36 that prevents fluid flow in the inflation lumen 30 unless the proximal end 14 is connected to a syringe or other means for inflating or deflating the balloon 32.

For urinary catheters such as Foley catheters, the catheter 10 is introduced into the patient and is advanced into the patient's urethra until the distal end 16 of the catheter 10, including the balloon 32, resides within the bladder. The balloon 32 is then inflated, typically by coupling a syringe to the proximal end 14 of the catheter 10 such that the syringe may communicate with the inflation lumen 30, and actuating the syringe to discharge fluid from the syringe, through the inflation lumen 30, and into the balloon 32. To remove a catheter 10, it is first necessary to deflate the balloon 32 anchoring the distal end 16 of the catheter 10. This is done by withdrawing fluid through the inflation lumen 30, typically through a syringe coupled to the inflation lumen 30 via inflation valve 36 and inflation port 34.

The balloon 32, which in one embodiment is made of an elastomeric material, is positioned around the catheter shaft. The balloon 32 is preferably engineered to retain its shape once inflated without significantly deforming due to pressures arising while within the body. The balloon 32 may include ribs (e.g., thicker polymer portions or added reinforcement) to ensure strength and symmetry of the material.

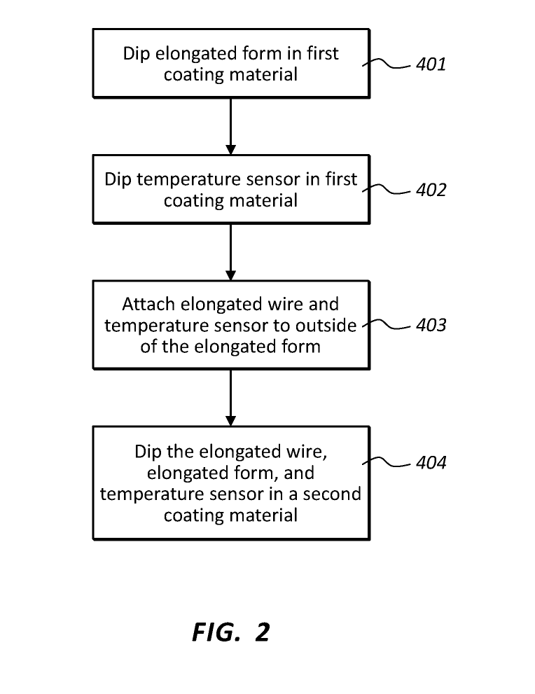

FIG. 2 describes a highly efficient method of manufacture that allows the formation of temperature-sensing catheters with a broad range of physical characteristics. The method involves manufacturing wireless temperature-sensing catheters reinforced with a metal element. The method of manufacturing a temperature-sensing Foley catheter described herein increases the quality and consistency of the catheter, as well as allowing the outer layers of the catheter to have broader material properties without an overcomplicated process.

In one embodiment, efficient measurement of a patient's temperature using the temperature of bodily fluid is accomplished by using a temperature sensor 20 embedded in a catheter 10 and transmitting the information wirelessly to an external display. A temperature sensor 20 may be embedded in a catheter 10 during the process of manufacturing the catheter, rather than embedding the temperature sensor 20 post-processing. A wireless temperature sensor 20 can be integrated into a catheter 10 to sense temperature inside the body without the need to connect wires. This leads to a completely embedded temperature sensor 20 that has no risk of patient contact.

The catheter 10 may be manufactured by dipping, for example by the methods described in U.S. Pat. No. 7,628,784, which is incorporated by reference in its entirety into this application. In one embodiment, in step 401, an elongated rod or “form” is dipped into a first liquid coating material to form a first layer of coating material on the form. The form has the shape and dimensions of the drainage lumen 40 of the catheter 10. This first coating layer forms the first layer of the catheter 10. In step 402, the temperature sensor 20 is also separately dipped into a first liquid coating material. In step 403, once the first layer has dried, an elongated wire is attached longitudinally to the outside of the first layer. In step 404, the form with first layer, temperature sensor 20, and an elongated wire (used to form the inflation lumen 30) is then dipped into a second coating material to form a second layer.

Alternatively, the temperature sensor 20 may be dipped only once, i.e., dipped only into the second coating without being first coated previously. Multiple dips into the second coating material may be necessary to form a second layer of appropriate thickness. The inflation eye 38 is then formed near the distal end 16 of the second layer to place the inflation lumen 30, formed by the elongated wire, in communication with the second layer. The second layer is then dried. Optionally, a third layer is applied with a subsequent dip and is dried.

The balloon 32 can be formed in a number of ways. In some preferred embodiments, the balloon 32 is formed by attaching a pre-formed balloon component to the second layer. In other embodiments, a masking material is applied to the exterior of the second layer in the balloon formation area such that upon dipping to form a third layer, a bond does not form between the second layer and the third layer in the balloon formation area near the inflation eye 38 of the inflation lumen 30. In such embodiments, the un-adhered portion of the third layer may form the balloon 32. Optionally, the form with first and second layers and the balloon formation layer is then dipped into another coating solution to form a third layer. Alternatively, no final layer may be used, e.g., the pre-formed balloon component or third layer used to form the balloon 32 forms the outermost wall of the balloon 32.

Once the third layer has dried, the catheter 10 is removed from the form. The space formerly occupied by the form and the elongated wire becomes the drainage and inflation lumens 40 and 30 (respectively). The balloon 32 can be inflated by infusing an inflation medium into an inflation port 44, through the inflation eye 38 of the inflation lumen 30 and into the balloon 32.

As discussed above, the catheter shaft beneath the balloon 32 may comprise two layers, a first layer and a second layer. Optionally, the first and second layers are formed from the same or similar material, typically latex or silicone, such that the resulting composite structure is essentially homogenous. It will be appreciated that the catheter shaft in some embodiments may comprise three layers, an inner layer, an intermediate layer, and an outer layer bonded to the outer surface of the intermediate layer.

The inflation lumen 30 runs parallel to the surface of the second layer until a point where the inflation lumen 30 is in fluid communication with the interior of the balloon 32 (e.g., at a point beneath the balloon 32). The portion that communicates with the interior of the balloon 32 is referred to herein as the inflation eye 38. At the proximate end of the catheter 10, the inflation lumen 30 branches off along branching arm 18 and terminates at the proximal end 14 of the catheter 10. A syringe engages the inflation valve 36 to infuse an inflation medium such as sterile water through the inflation lumen 30 to inflate the balloon 32.

Drainage eye(s) 42 are then formed (e.g., cut) in the distal end 16 of catheter 10 distal of the balloon 32, such that the drainage lumen 40 is in fluid communication with the drainage eye(s) 42. It should be appreciated that although a single drainage eye 42 is illustrated, it is contemplated that the tip 52 may include multiple drainage eyes 42.

In one embodiment, a wireless temperature sensor 20 is added mid-process to a catheter 10 as a single step instead of multiple post-processing steps to place a wireless temperature sensor 20 into a catheter 10 after manufacturing. As such, a purpose-built wireless temperature sensor 20 (e.g., a thin metal strip, film strip, circuit, wire, etc.), is integrated into the manufacturing process discussed above. It is carried through the rest of the Foley manufacturing process such that it is permanently integrated into the temperature-sensing Foley catheter 10.

The catheter 10 may be formed using a dip-coating process by dipping the wireless temperature sensor 20 and elongated form separately into a first coating material, and dipping the entire catheter 10, including the temperature sensor 20, elongated form, and an elongated wire in a second coating material, which coats both the entire inner and outer surfaces of the catheter 10 and causes the coating materials to be in direct contact with the surfaces. The catheter 10 may be coated with latex (most widely used among clinicians), red latex (stiffer and radiopaque), Silastic® material (firm but flexible, latex-based construction with smooth, nonstick silicone elastomer coating to reduce calcification build-up), or silicon, among other materials listed below. Catheter 10 may also be coated with an outer hydrogel coating to reduce friction, a major cause of irritation, and generally to improve patient comfort and safety. This is especially effective with latex and silicone catheters. A multiple-dip manufacturing process may be used to ensure a smooth surface with no excess material to cause irritation. Preferably, tip 52 is precisely molded to eliminate excess material that can cause irritation.

The following materials may be used in the manufacture of catheter 10: natural rubber latexes (available, for example, from Guthrie, Inc., Tucson, Ariz.; Firestone, Inc., Akron, Ohio; and Centrotrade USA, Virginia Beach, Va.), silicones (available, for example, from GE Silicones, Waterford, N.Y., Wacker Silicones, Adrian, Mich.; and Dow Coming, Inc., Midland, Mich.), polyvinyl chlorides (available, for example, from Kaneka Corp., Inc., New York, N.Y.), polyurethanes (available, for example, from Bayer, Inc., Toronto, Ontario, Rohm & Haas Company, Philadelphia, Pa.; and Ortec, Inc., Greenville, S.C.), plastisols (available, for example, from G S Industries, Bassett, Va.), polyvinyl acetate, (available, for example from Acetex Corp., Vancouver, British Columbia) and methacrylate copolymers (available, for example, from Heveatex, Inc., Fall River, Mass.). However, other materials not listed may also be used. Natural rubber latexes, polyurethanes, and silicones are preferred materials. Also, any combination of the foregoing materials may be used in making catheters. For example, an outer layer that includes latex and a methacrylate may be used with second and third layers that include latex but not methacrylate. Additionally, a polyurethane rubberize layer may used with latex second and third layers. Also, a polyvinyl acetate and latex rubberize layer may be used with latex second and third layers.

The above list of materials that can be used above in making catheters is not intended to be exhaustive and any other materials that can be used are within the scope of the invention. In addition, catheters 10 of the present invention are not limited to those having three layers of material. Any combination of layers can be used. For example, one or more additional coatings may be applied to the surface of the catheters 10 to provide lubricity, to reduce risk of infection, or for any other purpose.

Multiple types of wires are compatible with a catheter dipping process. A wire was tested using a resistor the same size as available temperature sensors 20 that meet current processing and use environments and specifications. In an exemplary embodiment, a fine copper wire that is coated (e.g., so as not to disrupt the latex) may be used. A coated wire may be effectively integrated into a latex dipping processes (i.e., can be coated in the latex dipping process) and is not detrimental to the solutions. Conformational coatings are also able to properly integrate into manufacturing by dipping. In an exemplary embodiment, an acrylic type of conformational coating may be used.

To ensure the ease of application of the temperature sensor 20 and flexibility of the catheter 10, a thin metal strip or film strip is preferred as the temperature sensor 20. The circuit is separated from the catheter 10 at sufficient distance from the catheter's 10 proximal end 14 to ensure it does not interfere with cutting equipment.

It is contemplated that the catheter 10 includes a temperature sensor 20 capable of wirelessly transmitting a signal derived from the temperature sensor 20 to a wireless receiver in an external display. A catheter 10 is engaged within the patient (e.g., the balloon is expanded in the bladder), and the catheter 10 includes a temperature sensor 20 that generates a signal representative of the patient's body temperature. Additional sensors may be used in addition to, or in lieu of, the temperature sensor 20 to detect and measure additional vital signs, for example sensors described in U.S. Publication No. 2013/0066166, which is incorporated by reference in its entirety into this application.

The temperature sensor 20 includes a wireless transmitter capable of wirelessly transmitting a signal representative of patient's temperature to the external display, which includes a receiver. Wireless temperature detection could occur in a variety of ways. In one embodiment, short range radiofrequency (RF) principles may be used. One short range RF protocols that can be used is Bluetooth technology. Wireless 802.11 communication principles may also be used.

Various methods can be used to power the circuit of the temperature sensor 20. In one embodiment, the temperature sensor 20 may be energized by a power source such as a small battery. One embodiment provides for an unpowered wireless temperature sensor 20 at the tip 52 of the catheter 10 and a secondary device attached to the patient's catheter 10 or abdomen in order to power the wireless temperature sensor 20 and detect temperature.

In one embodiment, the catheter 10 contains an unconnected, unpowered, and completely embedded circuit with the temperature sensor 20. The circuit extends from the distal end 16 to the proximal end 14 within the catheter 10. To power the wireless temperature sensor 20, a separate device is placed over the distal end 16 of the catheter 10 that can induce current into the circuit and measure the resistance/voltage drop across the circuit. This is similar to an radio-frequency identification (RFID) loop that is unpowered, but can be scanned and activated.

One embodiment provides for a powered circuit with a wireless temperature sensor 20 at the tip 52 of the catheter 10 and a circuit near the proximal end 14 of the catheter 10 with an antenna, which is battery powered and would last at least beyond the allowable use of the catheter 10. Other methods of powering the circuit, such as body heat, could also be used.

The wireless temperature sensor 20 could also communicate with other electronic medical record systems or have warnings about a patient's temperature to give clinicians feedback about a patient's health. Also, the catheter 10 could include on-board storage and data-logging of a patient's temperature for reading and identification at a later point in time.

The wireless temperature sensor 20 may interact with an external display, such as C. R. Bard Inc.'s CritiCore® Patient Monitoring System. This allows a clinician to accurately measure core body temperature and urine output without the expense or patient inconvenience of invasive temperature probes. Maintaining a normal core body temperature may result in fewer adverse outcomes—including an increased risk of surgical site infection, morbid cardiac events, ventricular tachycardia, wound infection and blood loss—with a resulting decrease in costs. Such a system can be used with a communication module to connect to a hospital's clinical information system for paperless management of vital signs. It should be appreciated that while sensing temperature is described, other vital signs, such as heart beat, breathing rate, and blood pressure, may also be measured.

FIG. 3 is side cross-sectional view of a catheter 10 with a deployed inflation lumen 30, and a braided section 50 of a reinforcement 54 extending from a balloon 32 to a proximal end 14 of the catheter 10. It should be appreciated that the temperature sensor 20 alternately may be embedded at different points along the distal end 16 of the catheter 10. In one embodiment, the temperature sensor 20 is located adjacent a drainage eye 42. In one embodiment, the temperature sensor 20 is located proximal the balloon 32 further down the catheter shaft. FIG. 3 illustrates an embodiment of the temperature sensor 20 located proximal the balloon 32, such that the inflation lumen 30, drainage lumen 40, and temperature sensor 20 are shown in cross-section. Closer to the drainage eye 42, a cross-section of the catheter 10 would not include the inflation lumen 30. Alternatively, various other locations for the temperature sensor 20 are possible.

The failure of a balloon 32 of a Foley catheter 10 to deflate represents a device failure that requires intervention. This is often related to inflation lumen 30 collapse. It can also be caused by pulling a vacuum on the inflation lumen 30 when trying to drain it too quickly. The present catheter 10 would prevent this situation entirely.

Since lumen collapse is generally the main cause of a non-deflating catheter, the inflation lumen 30 can be reinforced with a metal or plastic braid or coil. Preferably, any metal used is MRI compatible, such as MP35N, nickel-cobalt base alloy, and allows shaping the reinforcement 54, and catheter 10, with a thin profile. Kevlar, poly-paraphenylene terephthalamide, may also be used. The reinforcement 54 may be provided by a thin metal braid, although other materials are possible, such as shape memory alloys, etc. Shape memory alloys include copper-aluminum-nickel, copper-zinc-aluminum, and iron-manganese-silicon alloys. In one embodiment, the reinforcement 54 of the shaft is provided by a material, such as Nitinol, that imparts radial strength to the catheter body 12 to permit insertion without inflation lumen 30 collapse, but is soft and flexible after insertion (e.g., due to changing of properties due to temperature) to enhance patient comfort.

Catheter 10 with reinforcement 54 is believed to provide advantages with respect to, for example, maximizing drainage, ease of manufacture, ease of insertion, prevention of lumen collapse due to axial stiffness of catheter shaft, enhanced patient comfort, faster inflation and deflation times, etc.

With the catheter 10 in place, the risk of inflation lumen 30 collapse is significantly reduced. A reinforcement 54, such as a braided metal support, in the inflation lumen 30 for the prevention of inflation lumen collapse also resists collapse under vacuum conditions. Such a support would allow for the other layers of the catheter 10 to have broader material properties and still maintain consistent functionality. Previously, preventing lumen collapse has been accomplished with nylon-reinforced catheters. While a nylon braid or tube may be used, a thin metal braid is a preferred embodiment, as a metal braid is small enough to support the inflation lumen 30 without causing significant geometry changes to the catheter 10. A drainage lumen 40 with a metal braid support also easily integrates into the same process as catheter 10 dipping process outlined above. A metal-reinforced drainage lumen 40 would result in superior flow properties and resistance to kinking.

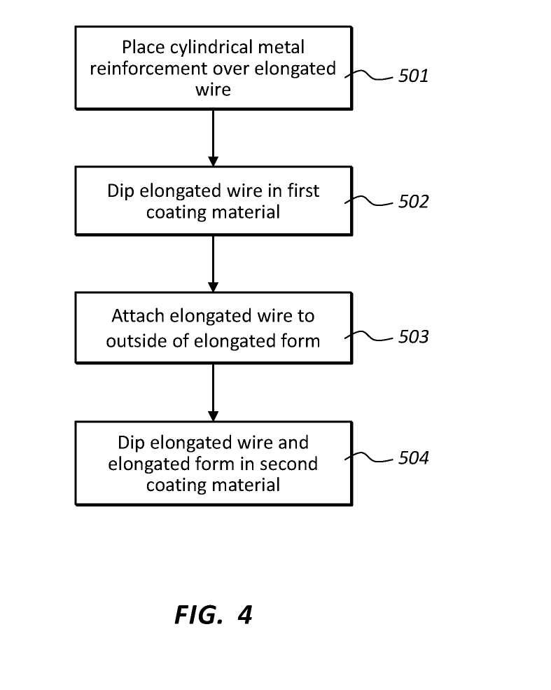

As illustrated in FIG. 4, the steps for manufacturing a catheter 10 with reinforcement 54 are similar to the manufacturing steps described above. However, in addition, in step 501, a cylindrical braided or coiled wire would be placed over an elongated wire used to form an inflation lumen 30 prior to dipping. The elongated wire would then be dipped in a first coating material in step 502. In step 503, the elongated wire would be attached longitudinally to the outside of a first layer separately formed on the elongated form used to form the drainage lumen 40. In step 504, the elongated wire and elongated form would be dipped in a second coating material. During the dipping process, the coating material integrates into the braid or coil and prevents the braid or coil from coming out of the catheter 10 upon removal of the elongated wire. It should be appreciated that the reinforcement section 50 may extend up to the inflation eye 38 or past it as long as a sufficient amount of water can pass through the braid or coil to allow inflation and deflation of the balloon 32.

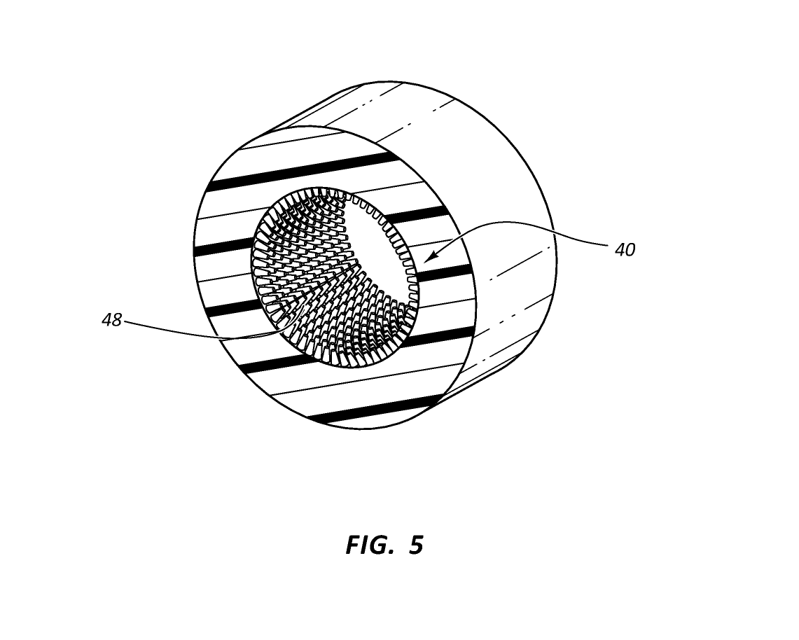

With regard to FIG. 5, to improve urine drainage through the catheter 10 and reduce urine surface tension on the lumen walls of catheter 10, the drainage lumen 40 of catheter 10 is preferably coated with a hydrophobic coating or treatment, and/or formed to include a patterned microstructure surface design, such as superhydrophobic patterned surface 48. This provides a better emptying mechanism and prevents fluid from being held for too long within the catheter 10. This also provides immediate fluid flow without columnating within the drainage lumen 40 and reduces unwanted fluid within the bladder and drainage lumen 40. Surface tension of the catheter 10 material (e.g., silicone) can cause the fluid passing through the catheter 10 to columnate instead of flowing continuously. Columnation can lead to the fluid (e.g., urine) backing up and not flowing properly though catheter 10. Columniation can leave residual fluid backed-up in the bladder, and leave residual fluid in the drainage lumen 40, which can lead to sanitation and health issues as well as errors in measurements of urine production and flow.

To prevent columnation, a hydrophobic coating or lubricious treatment may be added to the surface of the drainage lumen 40. Optionally, a patterned design can be used on the hydrophobic inner surface of the drainage lumen 40 to create superhydrophobic inner lumen surfaces and prevent columnation. The contact angles of a water droplet on a superhydrophobic surface may exceed 150° and the roll-off angle may be less than 10° making the superhydrophobic surface extremely difficult to wet. Superhydrophobicity can be obtained by artificially adding small-scale roughness to hydrophobic surfaces to keep droplets in a Cassie Baxter state, i.e., a state in which air remains trapped inside the microscopic crevasses below the droplet. The roughness of a surface decreases the wettability of hydrophobic surfaces resulting in an increased water-repellency. Wettability characteristics are those surface parameters which are directly linked to the wetting nature of materials; for instance, the contact angle is the angle the liquid droplet makes with the solid surface, and the surface free energy is the energy associated with the solid surface giving rise to the contact angle. Energetically the best configuration for the drop is on top of the corrugation like “a fakir on a bed of nails.”

Also, a droplet on an inclined superhydrophobic surface generally does not slide off; it rolls off. A benefit of this is that when the droplet rolls over a contamination, (e.g., dirt, dust, pollution, or viral/bacterial material, etc.) the contamination is removed from the surface if the force of absorption of the particle is higher than the static friction force between the particle and the surface. Usually the force needed to remove a particle/contamination is very low due to the minimized contact area between the particle/contamination and the surface. Accordingly, superhydrophobic surfaces have very good self-cleaning properties, and the growth of bacterial colonies is inhibited on the water repellant surfaces.

A superhydrophobic patterned surface 48, e.g., as shown in FIG. 5, may be formed on the surface of drainage lumen 40 such that liquid droplets will always be in the Cassie Baxter state, which improves the drainage and fluid flow inside the drainage lumen 40 and helps prevent columnation. Preferably, the superhydrophobic patterned surface 48 has a liquid/urine contact angle greater than 150° for extraordinary liquid/urine repelling properties and to eliminate the fluid columnating inside the catheter. Superhydrophobic patterned surface 48 may include tapered, cylindrical or squared microstructures (e.g., pillars) of a certain height and diameter and with a fixed pitch.

The superhydrophobic patterned surface 48 can be added to the surface by etching into the surface of a dipping form used to create the inner surface of the drainage lumen 40, or by adding an external flexible structure that is adhere to the dipping form before the catheter dipping process starts. Superhydrophobic surfaces could be fabricated from micro-arrays of RTV or any other type of polymer with pillars or posts pitches ranging from 450 to 700 microns. Preferably, the height of uniform pillars or post of a superhydrophobic surface is between 250 μm-500 μm, but the height can range as high as 800 μm. Optionally, UV cured silicone posts at 400 μm pitch fabricated by dispensing layers of adhesive on top of a flexible substrate can be used. In some embodiments, the posts or pillars have a diameter of between 50-175 μm. FIG. 5 shows an exemplary superhydrophobic patterned surface 48 formed on the entire inner surface of a drainage lumen 40. Although FIG. 5 shows the exemplary superhydrophobic patterned surface 48 as being on the entire inner surface of the drainage lumen 40, it is contemplated that the superhydrophobic patterned surface 48 may be on a portion of the inner surface of the drainage lumen 40.

One method of forming the microstructures (e.g., pillars or posts) of superhydrophobic patterned surface 48 is using a laser to form the inverse of the pattern/microstructures on the surface of a dipping form or mold that is then used to create the desired surface. Lasers can be used on the surfaces of many different materials ranging from ceramics, to metals, to polymers. Lasers have the ability to change both the surface dimensions (roughness and surface pattern) and the surface chemistry simultaneously which can then lead to a change in the wettability characteristics. Superhydrophobic patterned surfaces can also be prepared on a wide variety of surface shapes using a commercially available 3D printer for fabrication of large, complex polymer objects on a flat surface that later can be incorporated into the form, for the dipping process. This can be achieved where the micro-textured surface is monolithic with the body or flexible structure. The superhydrophobic behavior, such as the water column height supported, can be described by the same equations as those used to describe superhydrophobic behavior on surfaces with nano-scale textural features, thus eliminating the need for hydrophobic coatings.

The above embodiments have generally been described as being applied to a Foley catheter; however, the principles described may be applied to other types of catheters, e.g., angioplasty balloon catheters. Further, the features described in one embodiment may generally be combined with features described in other embodiments.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A catheter, comprising:
a balloon disposed near a distal end of the catheter proximal a tip formed at the distal end,
a drainage lumen extending from a drainage eye in a side wall of the tip to a proximal end of the catheter,
an inflation lumen extending from an inflation eye near the distal end in fluid communication with the balloon to the proximal end of the catheter, and
a temperature sensor disposed at the distal end of the catheter proximal the drainage eye, wherein the temperature sensor wirelessly transmits information representative of a patient's temperature to an external display.

2. A catheter, according to claim 1, wherein the inflation lumen further comprises a metal support.

3. A catheter, according to claim 1, wherein the temperature sensor wirelessly transmits information using Bluetooth or wireless 802.11 communication.

4. A catheter according to claim 1, wherein the temperature sensor communicates with the external display via a digital interface.

5. A catheter according to claim 4, wherein information is transmitted over the digital interface from the temperature sensor to the external display.

6. A catheter according to claim 1, wherein the temperature sensor is powered by a power source.

7. A catheter according to claim 6, wherein the power source is a small battery.

8. A catheter according to claim 6, wherein the power source is a patient's body heat.

9. A catheter according to claim 1, wherein the temperature sensor is powered by a secondary device attached to the catheter or a patient's abdomen.

10. A catheter according to claim 1, further comprising an unpowered circuit with a wireless temperature sensor that is powered by a circuit at or near the proximal end of the catheter where an antenna/power circuit loop is made and activated by a second device.

11. A catheter according to claim 1, wherein the catheter further comprises a powered circuit with a wireless temperature sensor and a battery-powered circuit near the proximal end of the catheter.

12. A method of manufacturing a catheter, the method comprising:
dipping an elongated form in a first coating material,
dipping a temperature sensor in a first coating material,
attaching an elongated wire and the temperature sensor longitudinally to an outside of the elongated form, and
dipping the attached elongated wire, elongated form, and temperature sensor together in a second coating material.

13. A catheter, comprising:
a balloon disposed near a distal end of the catheter proximal a tip formed at the distal end,
a drainage lumen extending from a drainage eye in a side wall of the tip to a proximal end of the catheter, and
an inflation lumen extending from an inflation eye near the distal end in fluid communication with the balloon to the proximal end of the catheter, wherein the inflation lumen is reinforced with a metal support.

14. A catheter according to claim 13, wherein the metal support comprises a braid or coil.

15. A catheter according to claim 14, wherein the metal support is selected from a group consisting of copper-aluminum-nickel, copper-zinc-aluminum, iron-manganese-silicon alloys, nickel-cobalt base alloy, or poly-paraphenylene terephthalamide.

16. A catheter according to claim 13, further comprising a wireless temperature sensor disposed at the distal end proximal the drainage eye.

17. A catheter according to claim 13, wherein the metal support extends from a point proximal the inflation eye to the proximal end of the catheter.

18. A catheter according to claim 13, wherein the metal support extends from a point distal the inflation eye to the proximal end of the catheter.

19. A method of manufacturing a catheter, the method comprising:
placing a cylindrical metal reinforcement over an elongated wire,
dipping an elongated form in a first coating material,
attaching the elongated wire longitudinally to an outside of the elongated form,
dipping the attached elongated wire and elongated form together in a second coating material.

20. A method according to claim 19, wherein the first coating material is integrated into the cylindrical metal reinforcement.
